(19) United States
(12) Patent Application Publication
(10) Pub. No.: US 2025/0262408 A1
(43) Pub. Date: Aug. 21, 2025

(54) HYPOTUBE PROXIMAL CONNECTION TO A HUB AND INTERLOCK TOOL FOR HANDLING A HYPOTUBE

(71) Applicants: STRYKER CORPORATION, Portage, MI (US); STRYKER EUROPEAN OPERATIONS LIMITED, Carrigtwohill (IE)

(72) Inventors: Arielle Wind Hassett, Salt Lake City, UT (US); Tammy Meichun Ho, Salt Lake City, UT (US); Kyler K. Hodgson, Salt Lake City, UT (US); Garrett Scott Janetzky, Salt Lake City, UT (US); Hannah Leigh Nyeboer, Cottonwood Heights, UT (US); Michael Troy Poor, San Jose, CA (US)

(73) Assignees: STRYKER CORPORATION, Portage, MI (US); STRYKER EUROPEAN OPERATIONS LIMITED, Carrigtwohill (IE)

(21) Appl. No.: 19/203,091

(22) Filed: May 8, 2025

Related U.S. Application Data
(63) Continuation of application No. PCT/US2024/029004, filed on May 10, 2024.
(60) Provisional application No. 63/507,705, filed on Jun. 12, 2023.

(51) Int. Cl. A61M 25/00

(52) U.S. Cl. CPC A61M 25/0097; A61M 25/0009; A61M 25/0045; A61M 25/0054; A61M 2209/08

(57) ABSTRACT

Medical devices comprising an elongated hypotube having an inner liner and an outer jacket, and a hub adhesively attached to a proximal end of the hypotube. A hub bond region at the proximal end of the hypotube at which the hub is adhesively attached has an open cut pattern which allows the inner liner to bond to the outer jacket through the open cut pattern that facilitates an effective bond between the hypotube and the hub and avoids air ingress as well as delamination of the outer jacket from the hypotube. A work holding device for securing a hypotube during processing. The work holding device includes a tube having an interlock junction on its distal end for coupling to a mating junction on a proximal end of the hypotube.

### RELATED APPLICATION DATA

The application is a continuation of International Patent Application No. PCT/US2024/029004, filed on May 10, 2024, which claims the benefit of U.S. Provisional Patent Application Ser. No. 63/507,705, filed on Jun. 12, 2023, the entire disclosures of all of which are hereby incorporated herein by reference in their entirety into the present application.

### FIELD OF THE DISCLOSURE

The field of the present disclosure generally relates to medical devices and methods for performing procedures within the vascular system of a patient, and more particularly, to hypotubes for use in intravascular devices such as catheters and the like, and methods and tools for manufacturing the same.

### BACKGROUND

Various designs of medical catheters have been previously provided for performing a variety of medical procedures, including interventional therapy, drug delivery, diagnosis, perfusion, and the like. In general, medical catheters are used by introducing the catheter through an entry site of a patient and into the vascular system of the patient, such as a vein or artery. The catheter is advanced from the entry site by guiding and pushing the catheter through the vascular system to a target site for performing a therapeutic and/or diagnostic medical procedure.

The catheter typically enters the patient's vasculature at a convenient location, such as a blood vessel in the neck or near the groin. Once the distal portion of the catheter (i.e., the portion farthest from the proximal handle of the catheter) has entered the patient's vascular system, the distal tip may be urged toward the target site by applying an axial force to the proximal portion of the catheter. Catheters having a relatively high level of pushability and kink resistance more effectively communicate this axial force.

Catheters frequently travel through the vascular system in a tortuous path, and are often required to change direction and to even double back on itself. The catheter may be “steered” by applying torsional forces to the proximal portion of the catheter. Catheters having a relatively high level of torqueability facilitate the steering process. Further, catheters having a relatively high level of flexibility can effectively conform to a tortuous vascular system of a patient.

The distance between the access site and the target site is often in excess of 100 cm. The inside diameter of the vasculature at the access site is often less than 5 mm. In view of the geometry of the patient's body, it is desirable to combine the features of torqueability, pushability, kink resistance, and flexibility into a catheter, which is relatively long and has a relatively small diameter. It is often desirable that the catheter have a relatively high level of pushability and torqueability, particularly near its proximal end. It is also desirable that a catheter be relatively flexible and steerable, particularly near its distal end. Further, it is sometimes desirable that the lumen of the catheter provide a pathway through the catheter having a low friction surface allowing other devices to pass through.

While conventional plastic catheters formed from polymer materials are relatively easy and inexpensive to manufacture, they may not provide sufficient pushability and torqueability, especially at very small diameters needed to access certain very narrow lumens such as the vessels of the neurovascular system. Accordingly, “hypotubes” have been utilized in catheters as forming all or most of the length of the catheter, as forming a portion of the catheter, and/or as reinforcing portions of a plastic catheter. A hypotube is a small diameter, thin-walled tube formed from metal or a metal alloy, such as stainless steel, nickel titanium alloy (e.g., nitinol), or the like. Hypotubes, being formed of metal, provide a number of desirable performance features for an intravascular catheter, including a relatively high level of pushability and torqueability, as well as kink-resistance. The thin wall of a hypotube permitted by the strength of the metal tube also has the advantage of providing a larger inside diameter (the lumen diameter) for a given outside diameter which allows larger structures to fit through the hypotube. For instance, the larger inside diameter of a hypotube may allow clot aspiration, the insertion and use of larger instruments, etc.

However, hypotubes may lack sufficient flexibility for navigating tortuous anatomical pathways, such as the vessels of the neurovascular system. Accordingly, hypotubes have been imparted with flexibility enhancing features such as openings (e.g., cuts) into or through the wall of the hypotube along the length of the hypotube. The openings may include circumferential, helical, longitudinal openings or other opening patterns which increase the flexibility of the hypotube. In order to seal the openings of the hypotube to provide a fluid sealed tube, and to provide other desirable characteristics to the hypotube (e.g., to provide smooth, low friction surfaces), thin liners and/or jackets are often applied to the hypotube. For instance, FIGS. 1A and 1B show a proximal portion of a typical prior art hypotube assembly 10. The hypotube assembly 10 includes a hypotube 12 having a plurality of cuts 11 for enhancing flexibility. The cuts 11 provides a ratio of open space (the holes in the tube wall created by the cuts) to tube wall of about 10% or less.

A polymer inner liner 14 is applied to the inside diameter of the hypotube 12 and a polymer outer jacket 16 is applied to the outside diameter of the hypotube 12. The polymer liners and/or jackets do not tend to bond to the hypotube 12, but instead simply form to the wall of the hypotube 12.

Furthermore, an elongated, flexible hypotube is typically one component of a catheter assembly which is attached to one or more other components of the catheter assembly. However, hypotubes do not easily and effectively bond to other components using adhesive. For instance, adhesives do not generally bond well to metal surfaces, adhesive tend to shrink during curing or heat bonding causing gaps and voids between the bonded parts, and the small geometries of catheter components also make it difficult to bond. In the case of a hypotube having an inner liner and/or outer jacket, adhesive bonding of catheter components may also cause delamination of the liners from the hypotube because the liners are not bonded to the hypotube.

For example, many catheter assemblies include a hub attached to the proximal end of the catheter for providing a handling structure for manipulating (e.g., applying axial forces (pushing and pulling) and rotational forces (torquing)) the catheter as it is advanced and steered to a target site through a patient's vascular system. The hub may also provide a channel through which other instruments are inserted and advanced through the catheter. The hub may also include one or more ports for introducing or removing fluids through the catheter. The hub must bond to the hypotube with a hermetic seal and with adequate tensile strength. The hub is typically attached to the proximal end of the elongated tube of the catheter using an adhesive. As shown in the example of FIGS. 2A and 2B, a proximal end of a hypotube assembly 10 (comprising the hypotube 12, inner liner 14 and outer jacket 16) which has been bonded to a hub 20 using a layer of adhesive 22 is depicted. The narrowness of the cuts 11 and low open space ratio (about 10%) results in no bond or only a minimal bond, between the inter liner 14 and outer jacket 16 through the cuts. As can be seen in FIGS. 2A and 2B, air gaps 18 tend to form between the outer surface of the hypotube assembly 10 (i.e., the outer surface of the outer jacket 16) and the inner surface of the hub 20. This is caused by the adhesive 22 shrinking while curing which creates an area of low pressure (vacuum) producing a pressure gradient which draws ambient air into the bond resulting in an air gap 18 in the adhesive bond. The low pressure can also cause delamination 26 of the outer jacket 16 from the hypotube 12. The air gaps 18 and delamination 26 can result in a faulty seal between the hypotube assembly 10 and the hub 20, and inadequate bond strength to withstand the pressures and forces encountered during use of the catheter.

A nother problem encountered in the process of manufacturing a catheter is securing and manipulating the relatively fragile hypotube during processing of the hypotube during manufacturing of a catheter. For example, a hypotube may need to be secured and manipulated during inspection, such as inspection after laser cutting the cuts 11, and while applying shrink fit sheaths, liners, jackets, and or attaching other components of a hypotube assembly or catheter assembly. Current hypotube processing techniques utilize a mandrel which is inserted into the lumen of the hypotube. FIG. 3 illustrates one currently available method for handling a hypotube 10 during processing. A loose fitting mandrel 30 is inserted through the central lumen of the hypotube 10. The mandrel 30 is then used to manipulate the hypotube 10 during processing, such as inspection and assembly. However, while the mandrel 30 does allow the hypotube assembly 10 to be moved axially using the mandrel 30 without manually touching the hypotube assembly 10 which risks damaging the hypotube assembly 10. However, the mandrel 30 does not allow rotation of the hypotube assembly 10 during inspection and processing.

Another method for handling a hypotube assembly 10 during processing is to use a support mandrel (not shown) with an interference fit. However, this procedure has a tendency to stretch and/or compress the flexibility enhancing struts of the hypotube deviating from strut design and resulting in a high rate of scrap (e.g., 40% scrap rate) due to deforming the hypotube out of acceptable design tolerances. Collets, clamps, and other work piece holding solutions will also not work due to the downstream process requirement of passing tight fit heat shrink tube over the connecting region of the hypotube.

Accordingly, improved hypotube-hub interface designs are needed to overcome the drawbacks of previous catheter designs. Furthermore, there is a need for better hypotube work holding devices for securing and manipulating a hypotube during the catheter manufacturing process.

### SUMMARY

Disclosed herein are medical devices comprising an elongated hypotube and a hub adhesively attached to a proximal end of the hypotube. A hub bond region at the proximal end of the hypotube at which the hub is adhesively attached has an innovative open cut pattern which facilitates an effective and strong bond between the hypotube and the hub which avoids delamination of the inner layer and/or outer jacket and air gaps in the adhesive. This results in a hub bond with a hermetic seal and strong tensile strength. In various aspects, the elongated medical device may be a catheter, an intravascular catheter, a neurovascular catheter, a laparoscopic medical device, or the like.

Accordingly, in one aspect, an elongated medical device comprises a hypotube having a tube wall extending from a proximal end to a distal end. The hypotube also has a hub bond region at the proximal end of the hypotube. The hub bond region includes a pattern of openings through the tube wall. For example, the pattern of openings may be cut into the tube wall, such as laser cut, etched, etc. The pattern of openings provides a ratio of open space to tube wall of from 35-75%, or from 60-70%, or greater than 50%, or greater than 20%. The hypotube may also have a proximal opening pattern in the tube wall for a proximal region of the hypotube proximal of the hub bond region.

The hypotube also has an inner liner applied to an inside diameter of the hypotube. The inner liner may be a polymer liner, or any other suitable material. In yet another aspect, the hypotube also has a polymer outer jacket applied to the outside diameter of the hypotube. The inner liner and outer jacket are applied to the hypotube in a manner such that the inner liner and outer jacket bond to each other through the pattern of openings in the tube wall of the hypotube.

A hub is bonded to the hypotube. The hub comprises a hub body having a tubular lumen for receiving the proximal end of the hypotube. The proximal end of the hypotube is disposed in the tubular lumen of the hub. The hub is bonded to the hypotube using an adhesive applied in the hub bond region such that the adhesive flows through the openings and bonds an inner polymer layer of the adhesive within the tube wall to an outer polymer layer between the outer surface of hypotube and an inner wall of the tubular lumen.

In yet another aspect, the pattern of openings comprises openings having a width of from 0.003 to 0.02 inches (0.076 to 0.51 mm). Alternatively, the openings may have a width of 0.150 mm +/−0.2 mm.

In another aspect, the pattern of openings comprises a plurality of circumferential slits in the tube wall angularly spaced apart and axially spaced apart along the tube wall.

In yet another aspect, the pattern of openings comprises a plurality of helically oriented slits extending along the tube wall.

In another aspect, the pattern of openings comprises a plurality of circle shaped opening extending along the tube wall.

In another aspect, the pattern of openings comprises a plurality of axially oriented slits angularly spaced around the circumference of the tube wall and axially spaced along the tube wall.

In still another aspect, the hypotube comprises a pattern of flexibility enhancing openings in the tube wall distal of the pattern of openings and extending along a length of the hypotube, wherein the ratio of the open space to tube wall of the pattern of flexibility enhancing openings is less than 50%. In other aspects, the ratio of the open space to tube wall of the pattern of flexibility enhancing openings is less than 45%, or less than 40%, or less than 35%, or less than 25% or less than 20% or less than 15%.

In another aspect of the elongated medical device, a most proximal edge of the hypotube has a rook shaped pattern forming a circumferential square wave pattern. The rook shaped pattern provides extra polymer bonding area prior to proximal cut. This decreases the risk of instruments inserted through the lumen of the hypotube from delaminating the inner liner from the catheter.

Also disclosed herein are innovative work holding tools for handling a hypotube during a process for manufacturing a catheter, or other medical device, comprising the hypotube. The tools are work holding devices configured to secure and manipulate a hypotube during downstream processing of the hypotube during manufacturing of a catheter. In one aspect, a work holding device comprises a length of tube having a proximal end and a distal end. The tube has an outer diameter that is substantially the same (“substantially the same” means it is dimensionally within 5% of the other structure) as a product hypotube to be secured by the work holding device. A proximal end of the tube has an interlock junction configured to releasably mate with a mating junction on a proximal end of the product hypotube. The interlock junction is configured such that it can provide axial movement of a mated product hypotube in both a proximal direction and a distal direction, and also rotational movement of the mated product hypotube about a central longitudinal axis of the hypotube.

In another aspect of the work holding device, the interlock junction comprises a sawtooth shape on the proximal end of the tube, wherein the sawtooth shape has an axially locking prong configured to bear against a mating prong of the mating junction to allow the work holding device to pull the product tube in the proximal.

In another aspect, the interlock junction comprises an interlocking key shaped prong configured to couple to a mating key shaped prong of the mating junction.

In still another aspect, the tube has a crimp section for crimping the tube onto a mandrel inserted into the lumen of the tube. The crimped crimp section secures the work holding device to the mandrel such that the work holding device translates and rotates along with translation and rotation of the mandrel.

In another aspect, the tube has an inner diameter that is substantially the same as an inner diameter of the product hypotube.

The work holding device may be used during processing of a product hypotube by mating the interlock junction with the mating junction of the product hypotube. Then, the product hypotube may be manipulated for downstream processing, such as inspection and assembly with other components of a medical device, such as a catheter, using the work holding device. The work holding device avoids subjecting the product hypotube to forces, such as the forces caused by an interference fit with a mandrel, which can damage the product hypotube and cause the product hypotube to be scrapped.

Accordingly, aspects of the disclosure described herein provide innovative hypotube assemblies and tools for downstream processing of hypotubes which result in improved devices, more efficient manufacturing processes, and less damaged and scrapped material.

### BRIEF DESCRIPTION OF THE DRAWINGS

The foregoing, along with other and further aspects of the disclosure, will now be described in greater detail in the below detailed description, to be read in view of the accompanying figures, wherein like reference numerals refer to like elements and the description for like elements shall be applicable for all described aspects wherever relevant.

FIG. 1A is a partial side view of a prior art hypotube;

FIG. 1B is a cross-sectional view of the prior art hypotube of FIG. 1A;

FIG. 2A is a partial side view of a prior art hypotube and hub assembly;

FIG. 2B is a cross-sectional view of the prior art prior art hypotube and hub assembly of FIG. 2A;

FIG. 3 is side view of a prior art mandrel for handling a hypotube during processing of the hypotube;

FIG. 4 is a side view of a hypotube and hub assembly, according to one aspect disclosed herein;

FIG. 5 is a side, partial, cross-sectional view of the hypotube of FIG. 4;

FIG. 6A is a side view of another pattern of openings in a hub bond region of a hypotube, according to another aspect disclosed herein;

FIG. 6B is a side view of yet another pattern of openings in a hub bond region of a hypotube, according to another aspect disclosed herein;

FIG. 6C is a side view of still another pattern of openings in a hub bond region of a hypotube, according to another aspect disclosed herein;

FIG. 6D is a side view of yet another pattern of openings in a hub bond region of a hypotube, according to another aspect disclosed herein;

FIG. 7 is a side view of a work holding device and mating product hypotube, according to one aspect disclosed herein;

FIG. 8 is a side view of another interlock junction of a work holding device and mating junction of a product hypotube, according to another aspect disclosed herein;

FIG. 9 is a side view of still another interlock junction of a work holding device and mating junction of a product hypotube, according to another aspect disclosed herein.

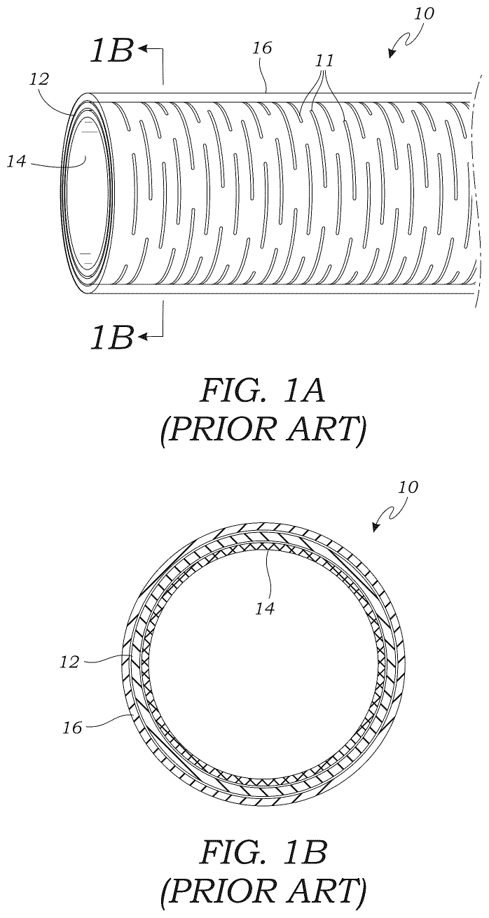

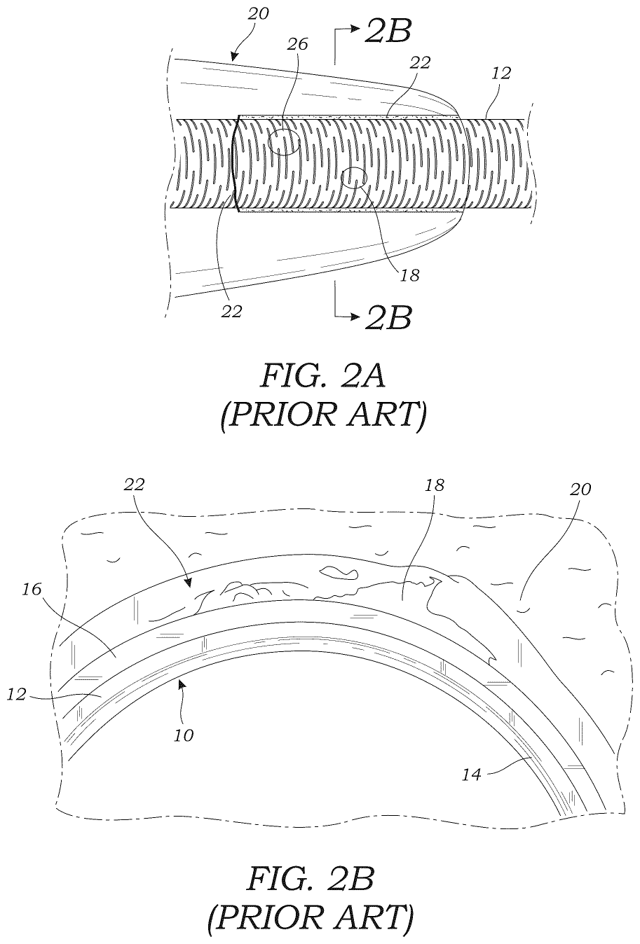

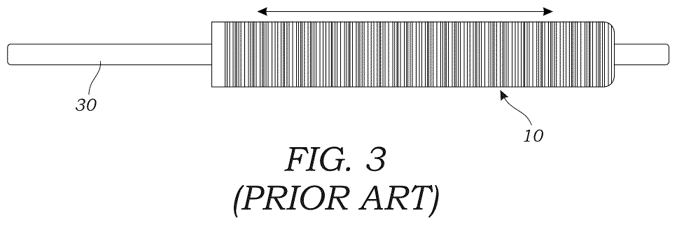

### DETAILED DESCRIPTION

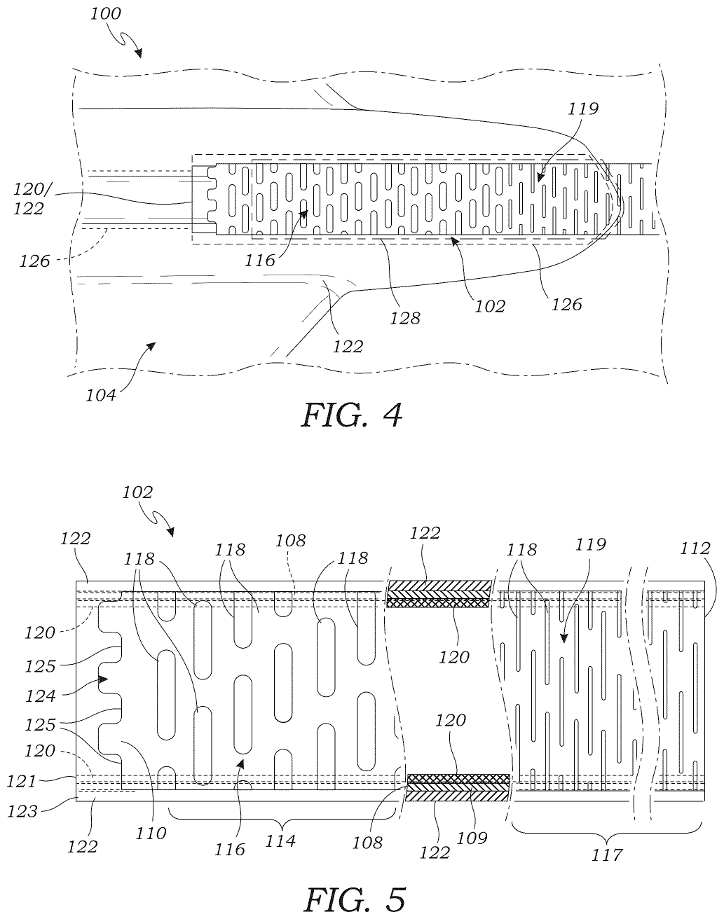

Turning now to the drawings, FIGS. 4-5 illustrate a medical device assembly 100 comprising a hypotube assembly 102 attached to a hub 104 using an adhesive 128. As shown in FIG. 5, the hypotube assembly 102 comprises a hypotube 108 having a tube wall 108 extending from a proximal end 110 to a distal end 112 of the hypotube 108. The hypotube 108 is formed of a metal or metal alloy. Some examples of suitable metals and metal alloys include stainless steel, such as 304V, 304L, and 316L stainless steel; nickel-titanium alloy such as a superelastic (i.e., pseudoelastic) or linear elastic nitinol; nickel-chromium alloy; nickel-chromium-iron alloy; cobalt alloy; tungsten or tungsten alloys; tantalum or tantalum alloys, gold or gold alloys, M P35-N (having a composition of about 35% Ni, 35% Co, 20% Cr, 9.75%. Mo, a maximum 1% Fe, a maximum 1% Ti, a maximum 0.25% C, a maximum 0.15% M n, and a maximum 0.15% Si); or the like; or other suitable metals, or combinations or alloys thereof

The proximal end 110 of the hypotube 108 has a hub bond region 114 having a hub bond region pattern 116 of openings 118. As shown in the aspects of FIGS. 4-5, the pattern 116 of openings 118 includes a plurality of rows of circumferentially arranged slits 118 angularly spaced apart. Each row of slits 118 may include 2, 3, 4, 5 or more angularly spaced apart slits 118. Each row of slits 118 is spaced apart axially along the longitudinal axis of the tube wall 109. Each row of slits 118 may also be angularly offset from the adjacent rows so that the slits 118 in one row are not angularly aligned with the respective slits 188 in the immediately adjacent rows. For example, the angular offset may form a helical pattern along the tube wall 108, or any other suitable pattern. Each of the slits 118 may have a width of from 0.003 to 0.02inches (0.076 to 0.51 mm), or a width of 0.150+/−0.2 mm, a length of about 5 to 15 mm. The slits 118 in each row may be spaced apart by about 0.18 mm+/−0.5 mm, and each row may be axially spaced apart from the adjacent rows by about 0.175+/−0.5 mm. The openings 118 are configured to be large enough to allow an inner liner 120 and an outer jacket 122 laminated on opposite sides of the hypotube 108 to bond to each other through the openings 118, as described below. The pattern 116 of openings 118 may be formed in the tube wall 109 by any suitable method, including, for example, cutting, laser cutting, etching, etc. The pattern 116 of openings 118 provides a ratio of open space to tube wall 109 of from 35-75%.

The hypotube 108 also has a distal opening pattern 119 in the tube wall 109 for a distal region 117 (or second portion 117) of the hypotube 108 distal of the hub bond region 114. The distal opening pattern 119 is provided as a flexibility enhancing feature, and the hypotube 108 is typically more rigid in this region to provide pushability and handling. The distal opening pattern 119 comprises a plurality of openings 115 which are substantially narrower than the openings 118, such that the openings 115 allow only very minimal or no bonding of the inner liner 120 to the outer jacket 122 through the openings 115 during the lamination process described below. The transition from the pattern 116 of openings 118 to the distal opening pattern 119 along the length of the hypotube 108 may be contained within the hub bond region 114 in which the hub 104 is bonded to the hypotube 108. This transition being contained with the more rigid hub/adhesive region prevents a kink point in the hypotube 108.

The most proximal edge of the proximal end 110 of the hypotube 108 has a proximal end pattern 124. In the illustrated aspect, the end pattern 124 is a rook shaped pattern forming a square wave pattern around the circumference of the proximal edge. As described below, the end pattern 124 includes open areas 125 which function as extra polymer bonding areas between the inner layer 120 and the outer jacket 122 of the hypotube assembly 102. The proximal end pattern 124 may be formed on the hypotube 108 by any suitable process, including for example, cutting, laser cutting, etching, etc. The proximal end pattern 124 has a length of about 0.612 mm or in the range of 0.3 mm to 1.3 mm (i.e., the height of the square wave pattern), from 4 to 12 peaks and associated open areas (valleys) around the circumference, and a ratio of open area (valley) to peak width ratio ranging from 0.5:1 to 3:1.

The hypotube assembly 102 also includes an inner liner 120 applied to an inside diameter of the hypotube 108 (i.e., the inside surface of the hypotube 108). The inner liner 120 may be a polymer liner, such as PTFE or other suitable material. The inner liner 120 may have a thickness of about 0.001 inches (0.025 mm) or in a range of from 0.00050 to 0.0050 inches (0.013 to 0.13 mm). For example, the inner liner 120 may be applied to the inside diameter of the hypotube 108 by dragging the liner through the lumen of the hypotube 108 and using PTFE beading to expand the inner liner 120 and press it against the inside diameter of the hypotube 108 to laminate the inner liner 120 to the inside diameter of the hypotube 108. A proximal end 121 of the inner liner 120 extends proximally beyond the proximal end 110 of the hypotube 108 (i.e., proximally beyond the end pattern 124).

The hypotube assembly 102 also includes an outer jacket 122 applied to the outside diameter of the hypotube 108. The outer jacket 122 may be a polymer jacket, such as a PEBAX® (a thermoplastic elastomer comprising polyamide and polyether) and AESNO® (a nylon material) co-extruded jacket, or other suitable material. The outer jacket 122 is loaded onto the outer diameter of the hypotube 108 and laminated down onto the outer diameter of the hypotube 108. A proximal end 123 of the outer jacket 122 also extends proximally beyond the proximal end 110 of the hypotube 108 (i.e., proximally beyond the end pattern 124).

During lamination of the inner liner 120 and/or outer jacket 122 to the hypotube 108, the inner liner 120 and outer jacket 122 also bond to each other through the openings 118, in the open areas 125 of end pattern 124, and along the length of the inner liner 120 and outer jacket 122 extending proximally beyond the proximal end 110 of the hypotube 108. The proximal end of the laminated inner liner 120 and outer jacket 122 may be trimmed to length after lamination. The open areas 125 of the end pattern 124 creates extra bonding area for the inner liner 120 to bond to the outer jacket 122 thereby decreasing the risk that materials inserted through the lumen, formed by the bonded inner liner 120 and outer jacket 122, will delaminate from each other.

Turning back to FIG. 4, the hypotube assembly 102 is assembled to the hub 104 by inserting the proximal end 110 of the hypotube assembly 102 into a tubular hub lumen 126 of the hub 104. An adhesive 128 is applied between the inner wall of the hub lumen 126 and the outer surface of the outer jacket 122 of the hypotube assembly 102. As the adhesive 128 cures to bond the hypotube assembly 102 to the hub 104, the adhesive 128 contracts and creates areas of low pressure (vacuum) tending to pull the outer jacket 122 toward the inner wall of the hub lumen. It is believed that the outer jacket 122 flexes to accommodate the pressure gradients thereby preventing the pressure gradient from drawing air into the adhesive bond 128 and averting air gaps from forming in the adhesive bond 128. This results in a stronger and more consistent bond between the hypotube assembly 102 and the hub 104. Moreover, the bond between the outer jacket 122 and the inner liner 120 through the openings 118 prevents the outer jacket 122 and inner jacket 120 from delaminating from the hypotube 108.

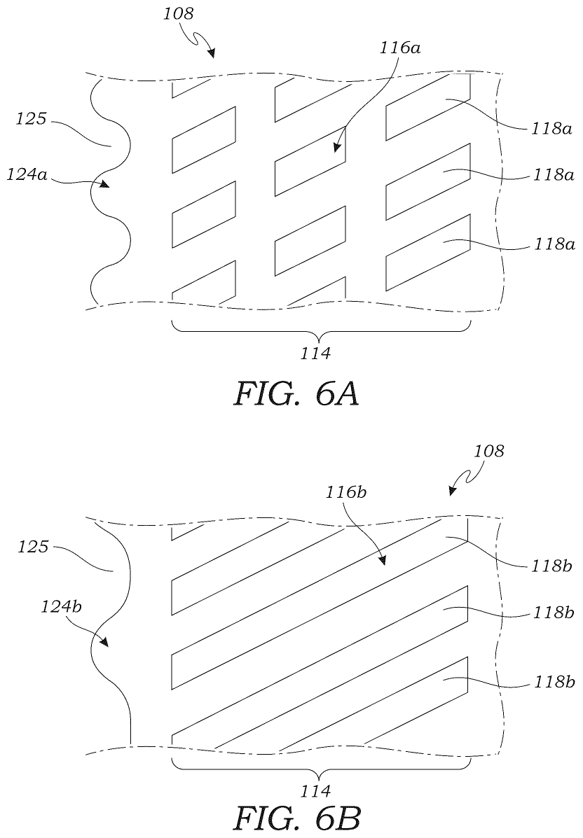

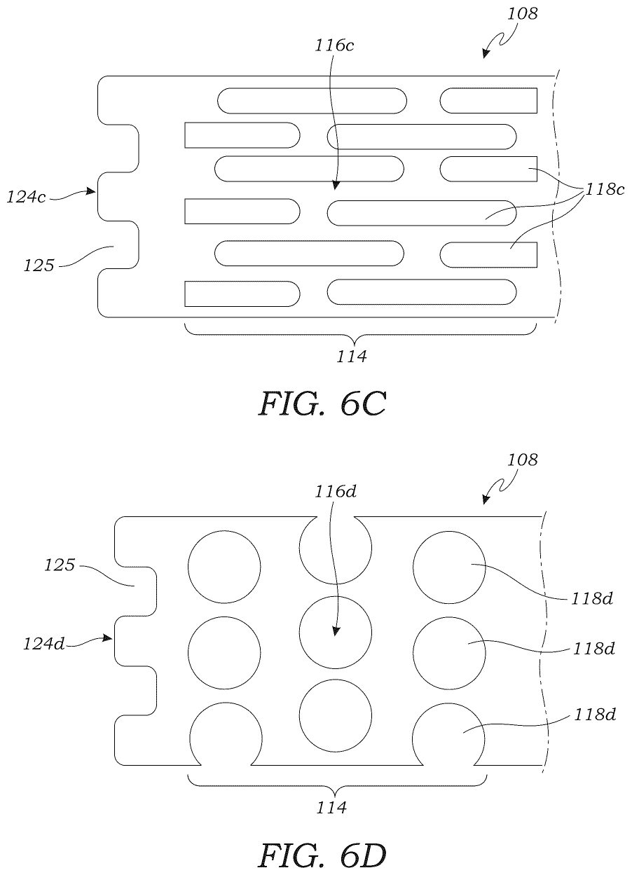

Turning now to FIGS. 6A-6D illustrate several alternative hub bond region patterns 116 and proximal end patterns 124 for the hypotube 108. FIG. 6A shows a portion of a hub bond region pattern 116a of openings 118a comprising a plurality of short angled openings 118a arranged in a plurality of helical patterns along the length of the hub bond region 114. FIG. 6A also shows a portion of proximal end pattern 124a comprising a sine wave shape having a short wavelength around the circumference of the proximal edge of the hypotube 108. FIG. 6B shows a portion of a hub bond region pattern 116b of openings 118b comprising a plurality of long angled openings 118b (longer than the short angled openings 118a) arranged in a plurality of helical patterns along the length of the hub bond region 114. FIG. 6B also shows a portion of a proximal end pattern 124b comprising a sine wave shape having a long wavelength (longer wavelength than the wavelength of the pattern 124a) around the circumference of the proximal edge of the hypotube 108. FIG. 6C shows a portion of a hub bond region pattern 116c of openings 118c comprising a plurality of axial oriented slits 118c having a long dimension oriented parallel to the longitudinal axis of the hypotube 108. FIG. 6C also shows a portion of a proximal end pattern 124c comprising a rook shaped pattern forming a square wave pattern around the circumference of the proximal edge of the hypotube 108. FIG. 6D shows a portion of a hub bond region pattern 116d of openings 118d comprising a plurality of circular openings 118d arranged in a plurality of rows of circular openings 118d extending along the length of the hub bond region 114. FIG. 6D also shows a portion of a proximal end pattern 124d comprising a rook shaped pattern forming a square wave pattern around the circumference of the proximal edge of the hypotube 108. Each of the openings 118a-118d is large enough, and is configured, to allow the inner liner 120 to laminate to the outer jacket 122 through the openings 118a-118d. Similar to the proximal end pattern 116 of the hypotube 108 of FIGS. 4-5, the proximal end patterns 116a-116d each provide a ratio of open space to tube wall 109 of about 40% or within the range of from 35-75%. This allows the inner liner 120 and outer jacket 122 to bond to each other through the openings 118a-118d, respectfully, to prevent air gaps in the adhesive bond 128 between the hypotube assembly 102 and the hub 104, as described herein.

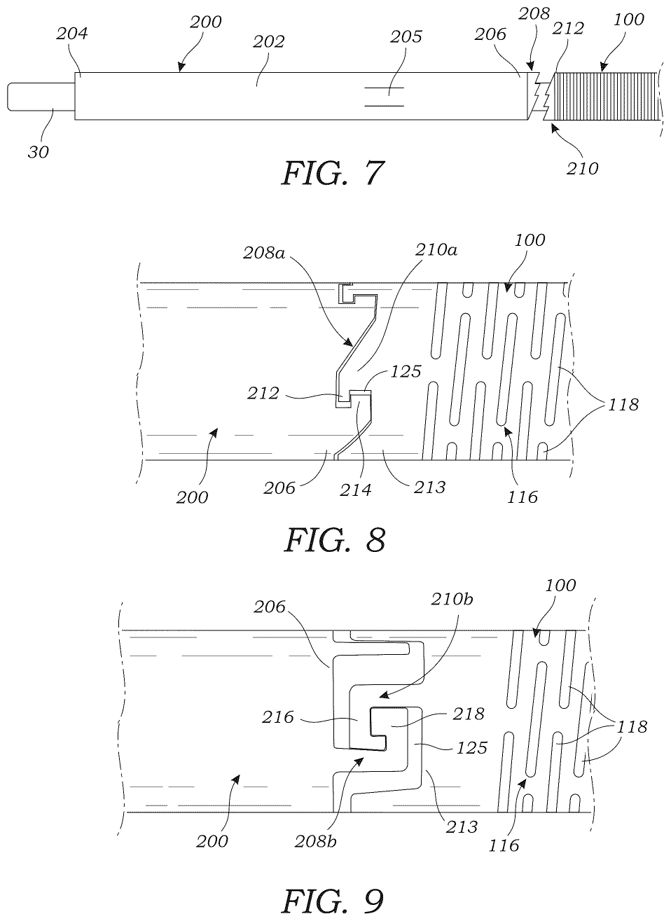

Referring now to FIG. 7, another aspect disclosed herein is directed to a work holding device 200 for handling a hypotube 100 (also referred to a product hypotube 100) during the processing of the hypotube 100 and assembly of the hypotube 100 into a catheter or other medical device comprising the hypotube 100. The work holding device 200 includes a cylindrical tube 202 having a proximal end 204 and a distal end 206. The tube 202 has an outer diameter that is substantially the same as the outer diameter of the product hypotube to be secured by the work holding device 200. The distal end 206 of the tube 202 has an interlock junction 208 configured to releasably couple to a mating junction 210 on the proximal end 213 of the product hypotube 100. The interlock junction 208 is configured such that it can impart axially force to the mated product hypotube 100 to hold the mated product hypotube 100 against axially forces in both a proximal direction and a distal direction and/or move the mated product hypotube 100 axially in both the proximal and distal direction, and also to impart torque on the mated product hypotube 100 to rotate the hypotube 100 about a central longitudinal axis of the hypotube 100.

The tube 202 also has a crimp section 205 for crimping the tube 202 to a mandrel 30 inserted into the lumen of the tube 202. The crimped crimp section 205 secures the work holding device 200 to the mandrel 30 such that the work holding device 200 translates and rotates along with translation and rotation of the mandrel 30.

As shown in FIG. 7, the interlock junction 208 comprises a sawtooth shape on the distal end 206 of the tube 202, and the mating junction 210 on the proximal end 213 of the hypotube 100 comprises a mating sawtooth shape which mates with the interlock junction 208. The interlock junction 208 may include an axially locking prong (not shown) configured to bear against a mating prong (not shown) of the mating junction 210 to allow the work holding device 200 to pull the product hypotube 100 in the proximal direction.

Referring to FIG. 8, another aspect of an interlock junction 208a and corresponding mating junction 210a is illustrated. The interlock junction 208a and mating junction 210a comprise interlocking key shapes which fit together by a combination of rotation and axially movement toward each other until an interlock tooth or prong 214 of the interlock junction 208a couples with a mating tooth 212 of the mating junction 210a. The interlock tooth 214 coupled to the mating tooth 212 provides bearing surfaces that allow the work holding device 200 to counteract axial forces tending to pull the hypotube 100 distally and to pull the hypotube 100 in a proximal direction.

Referring to FIG. 9, another aspect of an interlock junction 208b and mating junction 210b is shown. The interlock junction 208b and mating junction 210b are similar to interlock junction 208a and mating junction 210a, except that the former interlocks to allow work holding device 200 to torque the product hypotube 100 in both rotational directions (i.e., clockwise and counterclockwise). The interlock junction 208b includes an interlock tooth or prong 218 that bends around about 180 degrees and the mating junction 210b includes a mating tooth or prong 216 which bends around about 180 degrees in the opposite direction to the interlock tooth 218 such that the interlock tooth 218 mates to the mating tooth 216 in a manner which provides corresponding bearing surfaces allowing the interlock junction 208b to pull and push the mating junction 210b and hypotube 100 in both the proximal and distal directions, respectively.

The mating junctions 210, 210a and 210b also function as end patterns 124 having the open areas 125 and thus provide extra polymer bonding areas between the inner layer 120 and the outer jacket 122 of the hypotube assembly 102, as described above. Accordingly, any of the hypotubes 100 described herein may include the interlock junction 208 feature and may be processed using the same techniques using the work holding devices 200 disclosed herein.

The work holding device 200 may be used during processing of a product hypotube 100 by mating the interlock junction 208 with the mating junction 200 of the product hypotube 100. The product hypotube 100 may be manipulated for downstream processing, such as inspection and assembly with other components of a medical device, such as a catheter, using the work holding device 200. Accordingly, the work holding device 200 avoids subjecting the product hypotube 100 to forces and manual handling which can damage the hypotube 100, such as the forces caused by an interference fit with a mandrel or manual mishandling, which can damage the product hypotube 100 causing it to be scrapped.

Although particular aspects have been shown and described, it is to be understood that the above description is not intended to limit the scope of these aspects. While variations of the many aspects of the disclosure have been disclosed and described herein, such disclosure is provided for purposes of explanation and illustration only. Thus, various changes and modifications may be made without departing from the scope of the claims. For example, not all of the components described in the disclosure are necessary, and the devices disclosed herein may include any suitable combinations of the described components, and the general shapes and relative sizes of the components of the devices may be modified. Accordingly, aspects are intended to exemplify alternatives, modifications, and equivalents that may fall within the scope of the claims. The disclosure, therefore, should not be limited, except to the following claims, and their equivalents.

What is claimed is:

1. A medical device, comprising:
an elongated hypotube having a tube wall extending from a proximal end to a distal end, the hypotube having a hub bond region proximate the proximal end of the hypotube, the hypotube having an inside diameter and an outside diameter;
the hub bond region having a pattern of openings through the tube wall, the pattern of openings providing a ratio of open space created by the openings to tube wall of from 35-75%;
an inner liner applied to the inside diameter of the hypotube extending in the hub bond region of the hypotube, and an outer jacket applied to the inside diameter of the hypotube, the outer jacket extending in the hub bond region of the hypotube, the inner liner and outer jacket applied such that the inner liner and outer jacket bond to each other through the openings;
a hub having a hub body having a tubular lumen for receiving the proximal end of the hypotube;
the hub bonded to the hypotube using an adhesive applied to the hub bond region, thereby bonding the hub to the outer jacket of the hypotube.

2. The medical device of claim 1, wherein the pattern of openings comprises openings having a width of from 0.003 to 0.02 inches.

3. The medical device of claim 1, wherein the pattern of openings comprises openings having a width of from 0.130 mm to 0.170 mm.

4. The medical device of claim 1, wherein the pattern of openings comprises a plurality of circumferential slits in the tube wall angularly spaced apart and axially spaced apart along the tube wall.

5. The medical device of claim 1, wherein the pattern of openings comprises a plurality of helically oriented slits extending along the tube wall.

6. The medical device of claim 1, wherein the pattern of openings comprises a plurality of circle shaped opening extending along the tube wall.

7. The medical device of claim 1, wherein the pattern of openings comprises a plurality of axially oriented slits angularly spaced around the circumference of the tube wall and axially spaced along the tube wall.

8. The medical device of claim 1, wherein the hypotube comprises a pattern of flexibility enhancing openings in the tube wall distal of the pattern of openings and extending along at least a rigid portion of the hypotube, wherein the ratio of the open space to tube wall of the pattern of flexibility enhancing openings is less than 50% and the rigid portion of the hypotube is more rigid than the hub bond region having the pattern of openings.

9. The medical device of claim 1, wherein the hypotube comprises a pattern of flexibility enhancing openings in the tube wall distal of the pattern of openings and extending along a length of the hypotube, wherein the ratio of the open space to tube wall of the pattern of flexibility enhancing openings is less than 20%.

10. The medical device of claim 1, wherein a transition from the pattern of openings to the rigid portion of the hypotube is contained within the hub bond region in which the hub is bonded to the hypotube.

11. The medical device of claim 1, wherein a most proximal edge of the hypotube has a proximal end pattern which provides extra polymer bonding area between the inner liner and the outer jacket.

12. The medical device of claim 1, wherein the proximal end pattern comprises a rook shaped pattern forming a circumferential square wave pattern.

13. The medical device of claim 1, wherein the inner liner comprises a polymer material and the outer jacket comprises a polymer material.

14. The medical device of claim 1, wherein the inner liner is formed of PTFE and the outer jacket is formed of coextruded PEBAX® and AESNO.

15. A work holding device for securing and manipulating a product hypotube, the work holding device comprising:
a tube having a proximal end and a distal end, and having an outer diameter substantially the same as an outer diameter of the product hypotube;
the proximal end of the hypotube having an interlock junction configured to releasably mate with a mating junction on a proximal end of the product hypotube;
wherein the interlock junction is configured such that it can provide axial movement of a product hypotube mated to the interlock junction via the mating junction, and rotational movement of the mated product hypotube about a central longitudinal axis of the product hypotube.

16. The work holding device of claim 15, wherein the interlock junction comprises a sawtooth shape on the proximal end of the tube, wherein the sawtooth shape has an axially locking prong configured to bear against a mating prong of the mating junction to allow the work holding device to pull the product tube in a proximal direction.

17. The work holding device of claim 15, wherein the interlock junction comprises an interlocking key shaped prong configured to couple to a mating key shaped prong of the mating junction.

18. The work holding device of claim 15, wherein the tube has a crimp section for crimping the tube onto a mandrel inserted into the lumen of the tube.

19. The work holding device of claim 15, wherein the tube has an inner diameter that is substantially the same as an inner diameter of the product hypotube.

20. The work holding device of claim 15, wherein the interlock junction is configured to mate to the mating junction by a combination of rotating and translating the interlock junction relative to the mating junction.
